(19) United States
(12) Patent Application Publication
(10) Pub. No.: US 2022/0135617 A1
(43) Pub. Date: May 5, 2022

(54) CHROMATOGRAPHIC PURIFICATION OF AT LEAST ONE ENZYME SELECTED FROM A GROUP INCLUDING COLLAGENASE TYPE I, COLLAGENASE TYPE II, NEUTRAL PROTEASE AND CLOSTRIPAIN

(71) Applicant: NORDMARK PHARMA GMBH, UETERSEN (DE)

(72) Inventors: THOMAS SCHRÄDER, UETERSEN (DE); JÖRG LAMBRECHT, UETERSEN (DE); STEFAN DÖDING, UETERSEN (DE)

(73) Assignee: NORDMARK PHARMA GMBH, UETERSEN (DE)

(21) Appl. No.: 17/430,071

(22) PCT Filed: Feb. 14, 2019
(86) PCT No.: PCT/EP2019/053716

§ 371 Date: Aug. 11, 2021

(51) Int. Cl. C07K 1/20; C12N 9/52

(52) U.S. Cl. CPC C07K 1/20; C12Y 304/24003; C12Y 304/22008; C12N 9/52

(57) ABSTRACT

The present invention relates to a method for purifying at least one enzyme selected from the group consisting of collagenase type I, collagenase type II, neutral protease and clostripain from a mixture of substances, comprising as a method step at least one hydrophobic interaction chromatography, characterized in that, in the hydrophobic interaction chromatography, the stationary phase comprises a material selected from the group consisting of polypropylene glycol and butyl sepharose. The present invention further relates to the use of an enzyme thus purified for pharmaceutical, cosmetic and/or biochemical purposes.

### CROSS REFERENCE TO RELATED APPLICATIONS

This application is a national stage of International Application No. PCT/EP2019/053716 filed on Feb. 14, 2019, all of which is hereby incorporated by reference in its entirety.

### BACKGROUND

### Technical Field

This application relates generally to chromatography and more particularly to chromatographic purification of at least one enzyme selected from a group including collagenase type I, collagenase type II, neutral protease and clostripain.

### Discussion of Art

Clostridia are gram-positive, obligate anaerobic, spore-forming bacteria belonging to the family of clostridiaceae. The bacteria are widespread and occur everywhere, especially in soils and in the digestive tract of higher organisms.

Clostridium histolyticum, when cultivated on or in suitable nutrient media, secretes a complex mixture of enzymes containing collagenases, various other proteases, as well as low molecular weight components. The collagenases are subdivided into type I and type II (EC 3.4.24.3)—in short also collagenase I and collagenase II—depending on the converted substrate and have molecular weights between 115 and 125 kDa. Another component of the enzyme mixture secreted by clostridium histolyticum is the SH protease clostripain (EC 3.4.22.8), which occurs as a heterodimer and has a molecular weight of approximately 59 kDa. Clostripain specifically cleaves target proteins at arginine residues. Furthermore, the non-specific neutral protease with a molecular weight of approx. 34 kDa is secreted by Clostridium histolyticum.

Since all proteases are secreted by the bacterium into the medium, they can be easily separated from the cells in this way. In the natural environment, the proteases have the task of degrading tissue or fibrillar collagen and making the released peptides and amino acids available to the bacterium as a source of nutrients.

One of the commercial fields of application of collagenases is their use as a biochemical for in vitro isolation of cells from the tissue medium. In some cases, this cell isolation requires the other proteases neutral protease and/or clostripain in addition to collagenases. However, optimal results are only achieved if the proteases are present in specific ratios, depending on the respective intended cell isolation. The two collagenase types must also be present in certain ratios, depending on the respective application, in order to achieve optimal results.

Another field of application for the proteases from Clostridium histolyticum is their use as a biological agent for the production of pharmaceuticals, including, among others, the treatment of fibrotic cords in the palm and fingers in Dupuytren's disease, but also for various other diseases. Another pharmaceutical application of these proteases is their use in ointments for wound healing; for example, collagenases can be used for the enzymatic treatment of cutaneous ulcers. In addition to collagenases, the corresponding active ingredient also contains neutral protease and clostripain. Usual manufacturing processes for this active ingredient for wound treatment merely provide desalting, concentrating, and drying the cell-free culture supernatant. The enzymes are not separated in this process. The proportions of the enzymes in relation to each other are therefore determined by the fermentation and cannot be influenced by the current processes.

Since Clostridium histolyticum secretes a complex mixture of enzymes into the culture supernatant, it is necessary to separate the desired target enzymes and thus also to purify them from the culture supernatant. A method for the purification of enzymes from a culture supernatant of Clostridium histolyticum is known from DE 101 34 347 A1. However, a multi-stage purification process is provided, which exclusively uses chromatography materials based on styrene/divinylbenzene or ceramic hydroxyapatite. To purify the enzymes, a first chromatography step is required, which provides the use of a column filled with ceramic hydroxyapatite. This is followed by a second chromatography step consisting of anion-exchange chromatography with a styrene/divinylbenzene-based column matrix. This is followed by a third chromatography step, which also involves a styrene/divinylbenzene-based column matrix, this time in the context of cation exchange chromatography. In total, therefore, three chromatographic steps are necessary in this method, which make the purification of the enzymes from the culture supernatant very cost- and time-intensive.

Another method for the purification of collagenases from a liquid culture of the bacterium Clostridium histolyticum is described in US 2011/0070622 A1. The first step carried out is a protein precipitation by means of ammonium sulfate. This is followed by hydrophobic interaction chromatography as a second step, followed by a third purification step, which includes an anion exchange chromatography. This method again involves a material- and time-consuming sequence of several different method steps, with the result that only purified collagenases type I and type II are obtained, but no purified fractions of neutral protease and clostripain.

The purification processes described therefore place a strong focus on the two collagenases (type I and type II). To achieve the required purity of the collagenases, at least three purification steps are required in each case. Purification of neutral protease and clostripain is not described in these methods. For the separation of collagenase I and collagenase II, an additional separate chromatographic step is required in the known methods, which must be carried out specifically for this purpose. The material used for this purpose in the known methods is usually an anion exchanger.

The present invention is thus based on the object to avoid the disadvantages of the prior art. Preferably, in embodiments, an economical method for purifying at least one enzyme selected from the group including collagenase I, collagenase II, neutral protease and clostripain from a mixture of substances is to be provided. In particular, such a method for purifying at least one enzyme selected from a group that includes collagenase I, collagenase II, neutral protease and clostripain from the culture supernatant of Clostridium histolyticum is to be provided. Further, a material and time saving purification and separation of said enzymes is to be provided. It is also an object of embodiments of the present invention to provide a method by means of which the various enzymes can be separated from one another, so that they can subsequently be mixed in defined ratios. The separation of the enzymes from each other should preferably be carried out at high yield and in the required quality. A further object of embodiments is to reduce the number of steps required for purification of the enzymes from the culture supernatant of Clostridium histolyticum. In particular, in embodiments, it is an object of the present invention to reduce the number of chromatographic process steps for purification and/or separation of the enzymes.

### BRIEF DESCRIPTION

According to embodiments of the invention, the object described above is solved by a method for purifying at least one enzyme selected from the group including collagenase I, collagenase II, neutral protease and clostripain, from a mixture of substances, having as a method step at least one hydrophobic interaction chromatography (HIC), characterized in that, in the hydrophobic interaction chromatography, the stationary phase includes a material selected from the group including polypropylene glycol and butyl sepharose. In all embodiments of the present invention, it is further preferred that the stationary phase includes polypropylene glycol or butyl sepharose.

The separation principle of hydrophobic interaction chromatography is based on interactions of nonpolar surface regions of proteins with a hydrophobic stationary phase. These hydrophobic interactions are enhanced by an increased salt concentration in the solution serving as the mobile phase. The increased salt concentration thereby leads to a partial removal of the hydrate shells and thus to an exposure of hydrophobic regions of the proteins. These hydrophobic surface regions of the proteins now in turn interact with the hydrophobic residues of the stationary phase.

In the hydrophobic interaction chromatography, the stationary phase, i.e. the column material, usually consists of polymers that are chemically modified—i.e. hydrophobized—by specifically selected nonpolar functional groups. Here, the selectivity and capacity of the stationary phase depend on the selectivity and density of the functional groups used. In the present invention, it has proven particularly advantageous to use polypropylene glycol (PPG) or butyl sepharose as column material. For example, commercially available grade PPG-600M from Tosoh Bioscience LLC can be used as the polypropylene glycol column material. When butyl sepharose is used as column material, the commercially available Butyl Sepharose High Performance—Butyl Sepharose HP for short—and Butyl Sepharose Fast Flow—Butyl Sepharose FF for short—from GE Healthcare can be used. Butyl Sepharose HP is particularly preferred.

Butyl sepharose (also called “butyl agarose” or “cross-linked butyl agarose”) is a cross-linked agarose in which the hydrogen atoms of one part of the OH groups are replaced by 3-n-butoxy-2-hydroxypropyl residues (—CH₂—CHOH—CH₂—O—CH₂—CH₂—CH₃) and the OH groups concerned are thus correspondingly etherified. The degree of crosslinking is preferably 1 to 7%, particularly preferably 2 to 6%. The butyl sepharose is preferably present in the form of spherical particles with an average particle size in the range of 20 to 150 μm, particularly preferably in the range of 30 to 100 μm. For the separation of collagenase I and collagenase II from each other, an average particle size of 70 μm or less is preferred. An average particle size of 50 μm or less is particularly preferred for this purpose. The number of 3-n-butoxy-2-hydroxypropyl residues is preferably 10 to 200 μmol, particularly preferably 20 to 100 μmol and most preferably 30 to 70 μmol per milliliter of medium.

Due to the method according to embodiments of the invention, the purified enzymes can be obtained in high purity. The enzymes are very stable with respect to the hydrophobic interactions with the stationary phases polypropylene glycol and butyl sepharose as well as with respect to the buffer conditions of the mobile phase required for this purpose, and are subject to practically no decomposition. A particular advantage is that there is practically no denaturation of the enzymes to be purified, so that they are obtained in their native form and retain their biological activity. The purified enzymes therefore contain no or only very few denatured enzymes or parts thereof. This is very advantageous, since denatured enzymes are often very difficult to separate from the corresponding non-denatured enzymes, as they often have similar retention times in chromatography as the non-denatured enzymes. Due to the method according to the invention, the purified enzymes are therefore obtained in particularly good yield and in high purity. In the method according to the invention, the proteins to be purified thus essentially retain their native form and thus also their enzymatic activity. They can therefore subsequently be conveyed to their further use, such as in a pharmaceutical or biochemical composition, for which the retention of enzyme activity is essential. This is also of great importance with regard to the reproducible quality of an active pharmaceutical ingredient and the pharmaceutical preparations made from it.

With the method according to the invention it is further possible not only to purify the above-mentioned enzymes, collagenase I, collagenase II, neutral protease and clostripain, from impurities, but also to separate them from each other. Preferred is therefore also a method according to the invention which is characterized in that the mixture of substances comprises at least two enzymes selected from the group consisting of collagenase I, collagenase II, neutral protease and clostripain. Also preferred is a method according to the invention which is characterized in that the mixture of substances comprises at least two enzymes, wherein at least one enzyme is selected from the group consisting of collagenase I and collagenase II and at least one enzyme is selected from the group consisting of neutral protease and clostripain. Particularly preferred is a method according to the invention which is characterized in that the mixture of substances comprises at least three enzymes selected from the group consisting of collagenase I, collagenase II, neutral protease and clostripain. Very particularly preferred, however, is a method according to the invention which is characterized in that the mixture of substances comprises the enzymes collagenase I, collagenase II, neutral protease and clostripain.

The present invention provides a material- and timesaving and thus cost-effective method which is capable of producing and separating the enzymes, at high yield and in the required quality. This has the advantage that the composition of an active ingredient containing one or more of these enzymes can be varied as desired by selective mixing of the respective enzymes. This improves the quality of the active ingredient and leads to new active ingredients with a defined composition and better efficacy.

The enzymes collagenase I, collagenase II, neutral protease and clostripain are expressed and secreted by the bacterium Clostridium histolyticum. Since Clostridium histolyticum secretes a complex mixture of enzymes into the culture supernatant, there is a need to obtain these desired target enzymes by purifying them and separating them from each other. Most preferred, therefore, is a method according to the invention which is characterized in that the mixture of substances has been obtained from the culture supernatant of a culture of the bacterium Clostridium histolyticum. Equally preferred is a method according to the invention which is characterized in that the mixture of substances has been obtained from the culture supernatant of a culture of the bacterium Clostridium histolyticum and contains at least one enzyme selected from the group consisting of collagenase I, collagenase II, neutral protease and clostripain. Most preferably, the mixture contains at least two of the enzymes, most preferably at least three of the enzymes selected from the group consisting of collagenase I, collagenase II, neutral protease and clostripain. Most preferably, the mixture of substances contains all four enzymes.

The mixture of substances can be obtained, for example, from the culture supernatant of a culture of the bacterium Clostridium histolyticum by separating non-soluble components, for example by centrifugation or by filtering off cells, cell debris and other components that are non-soluble anyway. Further, for example, desalting, re-buffering and/or concentration or other method steps can be carried out, which are usually used for the preparation of culture supernatants.

Due to the method according to the invention, it is possible to purify both a single and several target proteins from a mixture of substances and, in particular, from the culture supernatant.

Surprisingly, it has been shown that the method according to the invention makes it possible to obtain the desired enzyme(s) from the culture supernatant in ready-to-use purity by only a single chromatographic process step. Preferably, the method according to the invention does not comprise any other chromatographic process besides the hydrophobic interaction chromatography on polypropylene glycol or butyl sepharose as stationary phase. Particularly preferably, the method according to the invention comprises only a single method step in which hydrophobic interaction chromatography is carried out with a stationary phase comprising a material selected from the group consisting of polypropylene glycol and butyl sepharose, and no further chromatography process besides this. This embodiment is hereinafter referred to as the “one-step process”. However, if necessary, further purification steps may follow or precede the chromatography.

With the method according to the invention, it is possible to purify and separate the above-mentioned enzymes, collagenase I, collagenase II, neutral protease and clostripain, from each other in a single chromatographic step. Since the developed method can provide the enzymes in the required quality in a one-step method, production costs are significantly reduced. First, significantly less material and time are required to carry out the method, and second, yields are much higher compared to established methods.

In addition, the one-step purification and separation also offers a distinct advantage in terms of the stability of the various proteases when purifying enzymes from mixtures of substances, in particular from culture supernatants. Indeed, the longer the mixture of enzymes, which are all proteases, is in the same mixture of substances and, in particular, in the same aqueous solution, the higher the risk and actual extent of mutual proteolytic degradation and thus irreversible destabilization and inactivation of the enzymes. This means that the faster the proteases are separated from each other as completely as possible, the higher the expected yield, the achievable purity and the stability of the purified individual enzymes. In particular, the storage stability is increased. This is also of great importance with regard to the reproducible quality of an active pharmaceutical ingredient and the pharmaceutical preparations made from it.

Preferred is a method according to the invention which is characterized in that, in the hydrophobic interaction chromatography, at least one aqueous solution is used as mobile phase that comprises at least one salt selected from the group consisting of ammonium sulfate and potassium chloride. When such mobile phases are used, the method can be carried out particularly effectively, and the advantages described herein become particularly apparent. In particular, the use of these mobile phases allows the four enzymes to be clearly separated from each other.

Particularly preferred is a method according to the invention which is characterized in that, in the hydrophobic interaction chromatography, the stationary phase comprises polypropylene glycol, and at least one aqueous solution comprising ammonium sulfate is used as mobile phase. This embodiment allows the four enzymes to be obtained in three separate fractions, wherein one fraction contains only the mixture of collagenases, i.e., a mixture of collagenase I and collagenase II, another fraction contains only the neutral protease, and the third fraction contains only the clostripain. This is advantageous because for many applications the mixture of collagenases is used without the need for their exact mixing ratio. At the same time, this embodiment of the method according to the invention can be carried out in a simple and material-saving manner. In this context, it is further preferred that the stationary phase consists of polypropylene glycol. Also preferred is therefore a method according to the invention in which the purified enzymes are obtained as a mixture of collagenase I with collagenase II in one fraction and neutral protease and clostripain separately in two further fractions.

Also particularly preferred is a method according to the invention which is characterized in that, in the hydrophobic interaction chromatography, the stationary phase comprises butyl sepharose, and at least one aqueous solution is used as mobile phase, which comprises at least one salt selected from the group consisting of ammonium sulfate and potassium chloride. Very particularly preferred is a method according to the invention which is characterized in that, in the hydrophobic interaction chromatography, the stationary phase comprises butyl sepharose, and at least one aqueous solution is used as mobile phase that comprises ammonium sulfate. Most preferred is a method according to the invention which is characterized in that, in the hydrophobic interaction chromatography, the stationary phase comprises butyl sepharose, and at least one aqueous solutions is used as mobile phase, containing potassium chloride. These embodiments allow the four enzymes collagenase I, collagenase II, neutral protease and clostripain to be obtained separately from each other in four separate fractions. For these embodiments, the use of a stationary phase comprising butyl sepharose is particularly preferred. Most preferred the stationary phase consists of butyl sepharose.

Thus, by using the present method, in addition to the purification of the enzymes, it is possible both to separate collagenases, neutral protease and clostripain from each other and, in addition to the separation of neutral protease and clostripain, to separate the two different types of collagenases (type I and type II) from each other. All these separations are possible in a one-step method.

The concentrations of ammonium sulfate and potassium chloride in the mobile phase have an influence on the hydrophobic interactions and thus on the binding properties to the respective stationary phase. The person skilled in the art can easily determine the amount of these salts necessary for purification and separation by preliminary experiments. Preferred is a method according to the invention which is characterized in that, in the hydrophobic interaction chromatography, at least one mobile phase is used which has a molar concentration of ammonium sulfate in the range from 0.3 to 1.5 mol/l and preferably in the range from 0.5 to 1.0 mol/l. Also preferred is a method according to the invention which is characterized in that, in the hydrophobic interaction chromatography, at least one mobile phase is used which has a molar concentration of potassium chloride in the range from 1.0 to 3.0 mol/l and preferably in the range from 1.5 to 2.5 mol/l.

The mobile phases used in the method according to the invention may also contain further components normally used in mobile phases, such as further salts. Examples of such salts are sodium chloride and calcium chloride. Insofar as the mobile phases contain sodium chloride, they preferably contain this in a molar concentration of 0.2 to 5 mol/l. If the mobile phases contain calcium chloride, they preferably contain this in a molar concentration of up to 50 mmol/l, particularly preferably up to 15 mmol/l. Furthermore, the mobile phases may contain tris(hydroxymethyl)-aminomethane (tris). Insofar as the mobile phases contain tris, they preferably contain this in a molar concentration of up to 60 mmol/l, particularly preferably up to 30 mmol/l. In addition, the mobile phases may contain organic solvents. These are preferably rather polar solvents. Particularly preferred are alcohols, especially isopropanol and/or polyols. Among the polyols most preferred are glycols, and among them, glycol and propylene glycol are again most preferred. Insofar as the mobile phases contain organic solvents, they preferably contain these in proportions of up to 50% by weight, preferably up to 40% by weight and particularly preferably up to 30% by weight. The pH of the mobile phase is preferably in the range of pH 6.0 to 9.5.

The mixture of substances to be separated is preferably applied to the stationary phase with the aid of a so-called application buffer. The composition and the properties of any application buffers used are the same as for the mobile phases.

The hydrophobic interaction chromatography is preferably carried out by means of a step elution, a gradient elution or a combination of these two. A method according to the invention is therefore particularly preferred which is characterized in that, in the hydrophobic interaction chromatography, a step elution, a gradient elution or a combination of these two is carried out. The step elution is particularly preferred for use on a production scale.

Preferred is further a method according to the invention which is characterized in that, in the hydrophobic interaction chromatography, a step elution, a gradient elution or a combination of these two is carried out, wherein at least three elution steps are carried out. Such a method is particularly well suited for separating collagenase I, collagenase II or the mixture of collagenases from neutral protease and clostripain. Particularly preferred is a method according to the invention which is characterized in that, in the hydrophobic interaction chromatography, at least three elution steps are carried out wherein the two collagenases, neutral protease and clostripain are separated from each other. Thus, the two collagenase types (type I and type II) are present here, mixed in a common fraction, while neutral protease and clostripain are present in two further fractions separate from each other and separate from the collagenases. Also particularly preferred is a method according to the invention which is characterized in that, in the hydrophobic interaction chromatography, at least three elution steps are carried out, wherein collagenase I, collagenase II, neutral protease and clostripain are separated from one another.

Also preferred is a method according to the invention, which is characterized in that, in the hydrophobic interaction chromatography, at least three elution steps are carried out in the form of a step elution, a gradient elution or a combination of these two, wherein the two collagenases (type I and type II), neutral protease and clostripain are separated from each other, or wherein collagenase I, collagenase II, neutral protease and clostripain are separated from each other. In the latter case, the additional separation of the two collagenases is preferably carried out by means of a linear gradient or by means of an additional elution step in the form of an elution step. The elution with at least three elution steps thus results in purification and separation of collagenases, neutral protease and clostripain in at least three value fractions.

Also preferred is a method according to the invention which is characterized in that, in the hydrophobic interaction chromatography, a gradient elution or a combination of gradient and step elution is carried out, wherein at least three elution steps are carried out. Particularly preferred is such a method according to the invention which is characterized in that the two collagenases, neutral protease and clostripain are separated from each other, so that collagenase type I and collagenase type II are present mixed in a common fraction, and neutral protease and clostripain are present in two further fractions separate from each other and separate from the collagenases. Particularly preferred is such a method according to the invention which is characterized in that collagenase type I, collagenase type II, neutral protease and clostripain are separated from each other.

### BRIEF DESCRIPTION OF THE DRAWINGS

FIG. 1: Chromatogram of the purification and separation of the concentrate of the culture supernatant on polypropylene glycol (PPG-600M) showing the fractions containing clostripain (F2-F5), collagenases (F6) and neutral protease (F7).

FIG. 2: MonoQ chromatogram of the collagenase value fraction (equivalent to fraction F6 in FIG. 1) after loading the polypropylene glycol (PPG-600M) column in HIC chromatography with different amounts (measured in volume-specific PZ activity according to Wunsch) of the concentrate of the culture supernatant.

FIG. 3: SDS-PAGE of the collagenase value fraction after separation on polypropylene glycol (PPG-600M) (equivalent to fraction F6 in FIG. 1). M: marker proteins; K: sample application concentrate (before purification/separation); Kol: Collagenase value fraction (F6).

FIG. 4: SDS-PAGE of the neutral protease value fraction after separation on polypropylene glycol (PPG-600M) (equivalent to fraction F7 in FIG. 1). M: marker proteins; K: sample application concentrate (before purification/separation); NPf: Neutral protease value fraction (F7) after chromatography; NPe: Neutral protease after concentration of F7 to lyophilized final product.

FIG. 5: Chromatogram of the purification and separation of the concentrate of the culture supernatant on butyl sepharose (Butyl Sepharose HP) showing the—compared to FIG. 1—additional separation of collagenase I and collagenase II.

FIG. 6: MonoQ chromatogram for comparative analysis of the respective collagenase value fractions after purification and separation of the cell-free, concentrated culture supernatant by HIC chromatography. A: Collagenase value fraction after separation on polypropylene glycol (PPG-600M) (procedure variant 1); B: Collagenase type II value fraction after separation on butyl sepharose (Butyl Sepharose HP) (procedure variant 2); C: Collagenase type I value fraction after separation on butyl sepharose (Butyl Sepharose HP) (procedure variant 2).

FIG. 7: SDS-PAGE for comparison of a currently commercially available collagenase (Ka) purified via several chromatographic steps with the collagenase (Kn) obtained in one chromatographic step according to method variant 2 of the invention. M: Marker proteins.

### DETAILED DESCRIPTION

The first sub-step in the hydrophobic interaction chromatography consists of applying the mixture of substances to be separated to the chromatographic column, preferably with the aid of a so-called application buffer. The application buffer is usually the specifically modified, highly saline aqueous matrix in which the mixture of substances dissolved therein accumulates for purification and separation, in the present method according to the invention, in particular in the context of the processing from the culture supernatant of a culture of the bacterium Clostridium histolyticum. In the extremely hydrophilic medium of the application buffer, very strong hydrophobic interactions of the proteins with the stationary phase occur, so that most proteins are almost completely fixed to the stationary phase.

As a second sub-step of the hydrophobic interaction chromatography—and thus before the elution of the value fractions with the target proteins, in the present method according to the invention with the enzymes collagenase I, collagenase II, neutral protease and/or clostripain—at least one so-called washing step is preferably carried out. In this washing step, the chromatography column including the proteins bound to the stationary phase is washed, wherein any impurities which are rather polar or hydrophilic and therefore not bound to the stationary phase, and which are often of low molecular weight, are washed out and thus separated from the target proteins. An aqueous buffer solution also containing a high salt content is usually used as the so-called wash buffer.

The composition of the application buffer and the wash buffer can differ. However, one and the same buffer solution can also be used both as an application buffer and as a wash buffer.

In the hydrophobic interaction chromatography, the washing step(s) are followed by the elution steps as further sub-steps. In particular with regard to the present method according to the invention, these are those sub-steps in which the value fractions with the target proteins are eluted from the column by successive adjustment of the composition of the mobile phase (elution buffer), in the case of hydrophobic interaction chromatography in particular by reducing the salt content of the mobile phase. This elution can be gradually carried out isocratically, i.e. in steps with constant composition of the mobile phase (step elution), as a gradient, i.e. with a targeted continuously changing composition of the mobile phase (gradient elution), or as a combination of step and gradient elution.

In certain constellations of hydrophobic interaction chromatography, one of the target proteins may show less strong hydrophobic interactions with the stationary phase than the remaining target proteins of the substance mixture and is therefore not bound to the stationary phase with the same intensity already in the application and wash buffer. In these cases, therefore, the wash buffer can under certain circumstances simultaneously serve as the first elution buffer, so that the same sub-step of the hydrophobic interaction chromatography represents in parallel both the wash step and the first elution step. In this case, the rather polar or hydrophilic, often low-molecular impurities are first washed out in the same sub-step and then the first value fraction is eluted with the less strongly bound target protein. In such constellations, the volume of the first elution buffer used—irrespective of whether it also functions as a wash buffer—can control the quality of separation of the target protein in question from the remaining target proteins of the mixture. In this case, the amount of mobile phase used is often expressed as the column volume (CV).

Accordingly, in the present method according to the invention, it is possible to control the content of clostripain in the corresponding eluted value fractions (elution fractions) via the volume of the first elution buffer (clostripain elution buffer) used, since clostripain shows less strong hydrophobic interactions with the stationary phase than the remaining target proteins of the mixture of substances (collagenase I, collagenase II and neutral protease). Thus, a large volume of the first elution buffer results in clostripain eluting completely or nearly completely in a separate, pure clostripain fraction. If, on the other hand, a smaller volume of the first elution buffer is used, the quantitative ratios of clostripain in the respective elution fractions shift. This then leads to the fact that not all or almost all of the clostripain is eluted in a separate fraction, but that the eluted clostripain is distributed both to a separate, pure clostripain fraction and to the respective subsequent collagenase fraction. Thus, the amount of the first elution buffer can be used to select whether clostripain should be eluted almost completely in a separate, pure clostripain fraction or whether part of the clostripain should be eluted as a component of the respective subsequent collagenase fraction and thus also occur together with collagenase(s) in one fraction. In the latter case, the choice of suitable salt concentrations of the mobile phase during the elution of the collagenases can control whether the second part of the clostripain accumulates together with both collagenases (type I and type II) in a common elution fraction (column material polypropylene glycol or butyl sepharose without additional separation of the two collagenase types) or together only with collagenase II in a common elution fraction (column material butyl sepharose with additional separation of the two collagenase types). The necessary volume of the mobile phase (of the first elution buffer) leading to an almost complete elution of the clostripain in a separate, pure clostripain fraction and thus to an almost complete separation of the clostripain from the collagenases and the neutral protease depends on the specific combination of different factors (stationary phase, mobile phase, dimensions of the column, etc.). However, preferred for separating the clostripain according to the invention is a method characterized in that the chromatography column is eluted with at least 10 column volumes, more preferably with at least 12 column volumes, and most preferably with at least 15 column volumes, of the first elution buffer. With such volumes of clostripain elution buffer, the clostripain is usually eluted completely or almost completely from the column.

The present method allows to obtain collagenase I, collagenase II and mixtures of these two types of collagenases in a purity of at least 80%, preferably of at least 90%, wherein the purity of the collagenases is determined by analytical anion-exchange chromatography (e.g. using a GE Healthcare MonoQ column with tris buffer as mobile phase at room temperature). A method according to the invention in which collagenase I, collagenase II or mixtures of these collagenases are obtained in a purity (determined as indicated above) of at least 80%, preferably at least 90%, is therefore preferred. Neutral protease can be obtained by the method according to the invention in a purity of at least 70%, preferably of at least 80%, wherein the purity of the neutral protease is determined by SDS-PAGE (according to Laemmli U. K.; Nature 227: 680-685, 1970). Also preferred, therefore, is a method according to the invention in which neutral protease is obtained in a purity (determined as indicated above) of at least 70%, preferably of at least 80%. Clostripain can be obtained by the method according to the invention in a purity of at least 60%, preferably of at least 70%, wherein the purity of the clostripain is determined by SDS-PAGE (according to Laemmli U. K.; Nature 227: 680-685, 1970). Also preferred, therefore, is a method according to the invention in which clostripain is obtained in a purity (determined as indicated above) of at least 60%, preferably of at least 70%. All of these purity values are purity levels that satisfy the requirements when these enzymes are used for most biochemical and medical purposes. Most preferred is a method according to the invention in which collagenase I, collagenase II or mixtures of these collagenases are obtained in a purity of at least 80% (determined as indicated above), neutral protease in a purity of at least 70% (determined as indicated above) and clostripain in a purity of at least 60% (determined as indicated above). Most preferred is a method according to the invention in which collagenase I, collagenase II or mixtures of these collagenases are obtained in a purity of at least 90% (determined as indicated above), neutral protease in a purity of at least 80% (determined as indicated above) and clostripain in a purity of at least 70% (determined as indicated above).

These enzymes are also obtainable in this purity in a one-step method.

Furthermore, a process according to the invention is preferred, which is characterized in that a linear flow rate of 100 to 300 cm/h, in particular of 150 to 250 cm/h, is used in the hydrophobic interaction chromatography. These flow rates result in optimal purification and separation of the enzymes.

Preferably, the method according to the invention does not comprise gel filtration steps and/or protein precipitation steps. Gel filtration is a very time-consuming method, which thus entails high costs and increases the risk of self-digestion of the enzymes to be separated. Protein precipitation steps, such as ammonium sulfate precipitation, can lead to undesirable structural changes in the proteins. Also, protein precipitation does not allow enzymes to be obtained with the high purity provided by the present method.

Preferably, at least one enzyme purified with the method according to the invention is used for pharmaceutical and/or biochemical purposes. Pharmaceutical purposes include any use of one or more of said enzyme(s) as an active pharmaceutical ingredient, wherein the use is not subject to any restriction as to indication, dosage form/preparation or mode of application. Furthermore, the enzyme(s) may be used for biochemical purposes, such as in vitro cell isolation.

Also preferably, at least one enzyme purified with the method according to the invention is used for cosmetic purposes. The use of the enzyme or enzymes thus also extends to the use for purely cosmetic purposes, i.e., a cosmetic use as distinguished from a therapeutic purpose.

The invention also includes an enzyme itself which is obtained by the method according to the invention. In addition, the invention also includes compositions comprising at least one enzyme obtained by the method according to the invention. The present invention further relates to the use of an enzyme purified with the method according to the invention for pharmaceutical, cosmetic and/or biochemical purposes.

After cultivation of clostridium histolyticum carried out in a suitable fermentation medium, the cells and other non-soluble components are separated from the culture supernatant, for example by centrifugation and/or filtration. The culture supernatant, which contains the enzymes collagenase I, collagenase II, neutral protease and clostripain, can be concentrated in the usual way before purification and separation of these proteins by hydrophobic interaction chromatography.

Hydrophobic interaction chromatography (HIC) is then performed to purify and separate the enzymes from the culture supernatant. For this purpose, the cell-free and, if necessary, suitably concentrated culture supernatant is applied, for example, to a chromatography column filled with polypropylene glycol (PPG) and/or butyl sepharose, wherein the enzymes—among other components—bind to the column material. After washing out unbound molecules, the elution of the initially bound components, including the target proteins, is carried out in a buffered system in the pH range of 6.0 to 9.5 at a linear flow rate of 100 to 300 cm/h by means of step elution, gradient elution, or a combination of these two, wherein the salt content is successively reduced. The elution of the proteins is preferably carried out in three or more elution steps. Here, three value fractions can be obtained, wherein one fraction contains the collagenases (type I and type II) together, another fraction contains neutral protease, and the third fraction contains clostripain (separation on PPG or butyl sepharose). In the case of chromatography on butyl sepharose, an additional separation of the two collagenases (type I and type II) from each other is possible. This separation is then preferably performed with a combination of step and gradient elution in at least three elution steps or with a four-step elution. Accordingly, four value fractions are obtained, where—as before—one fraction contains neutral protease and another fraction contains clostripain. However, the collagenases are now obtained separately in a third fraction containing collagenase I and a separate fourth fraction containing collagenase II.

The individual elution fractions can now be desalted and/or concentrated by means of conventional methods, such as a tangential flow filtration (TFF) process. Subsequently, the resulting material can be lyophilized by means of likewise customary methods, e.g. freeze-drying. If necessary, further purification steps can also follow.

A flow diagram of the entire process is shown in scheme 1 below. Depending on the variant used, this results in three or four different end products which are then available for the desired further use or further processing, in particular also a specific and defined mixture of several of these end products.

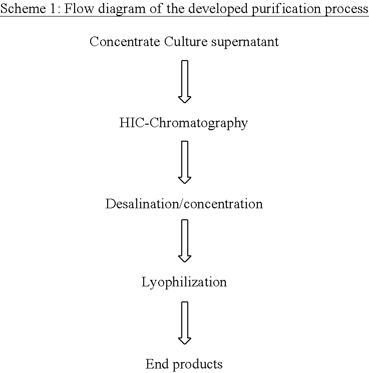

Depending on the stationary phase and mobile phase used (type and amount of salts in the mobile phase), this method can therefore achieve, for example, the following:

By using PPG as column material and ammonium sulfate (0.3-1.5 mol/l, in particular 0.5-1.0 mol/l) as salt, a separation of collagenases, neutral protease and clostripain is achieved, wherein the two types of collagenases (type I and type II) are present mixed in a common fraction, while neutral protease and clostripain are present in two further fractions separate from each other and separate from the collagenases. The use of butyl sepharose as column material with potassium chloride (1.0-3.0 mol/l, especially 1.5-2.5 mol/l) as salt allows an additional separation of the collagenases into collagenase I and collagenase II. The same is achieved when butyl sepharose is used as column material with ammonium sulfate (0.3-1.5 mol/l, especially 0.5-1.0 mol/l) as salt.

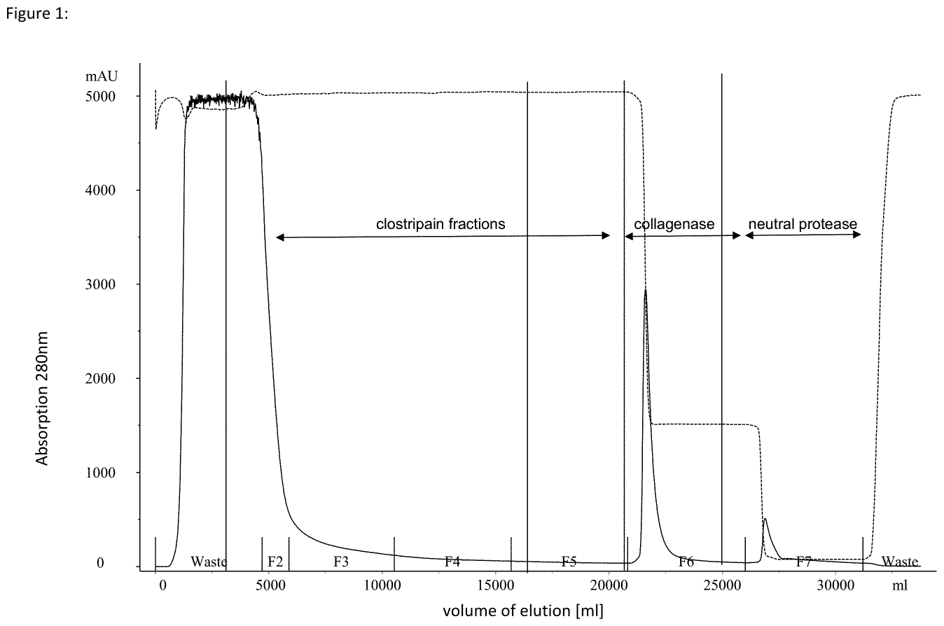

FIG. 1: Chromatogram of the purification and separation of the concentrate of the culture supernatant on polypropylene glycol (PPG-600M) showing the fractions containing clostripain (F2-F5), collagenases (F6) and neutral protease (F7).

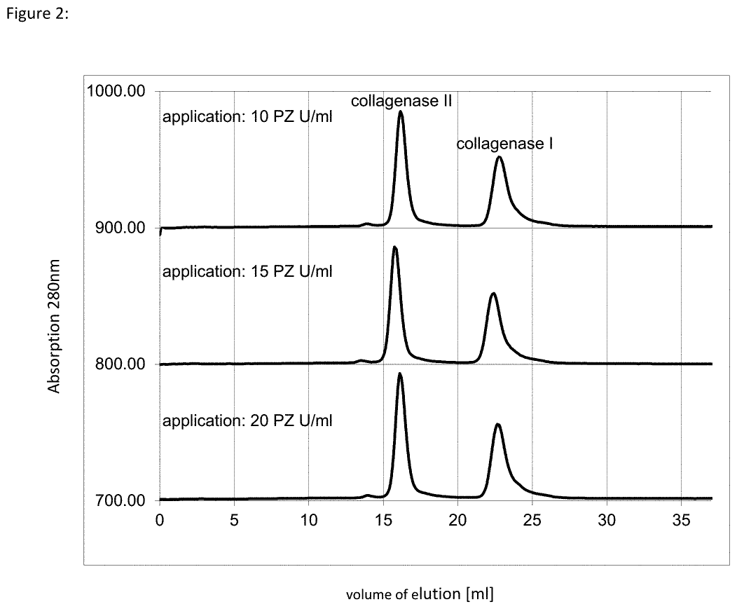

FIG. 2: MonoQ chromatogram of the collagenase value fraction (equivalent to fraction F6 in FIG. 1) after loading the polypropylene glycol (PPG-600M) column in HIC chromatography with different amounts (measured in volume-specific PZ activity according to Wunsch) of the concentrate of the culture supernatant.

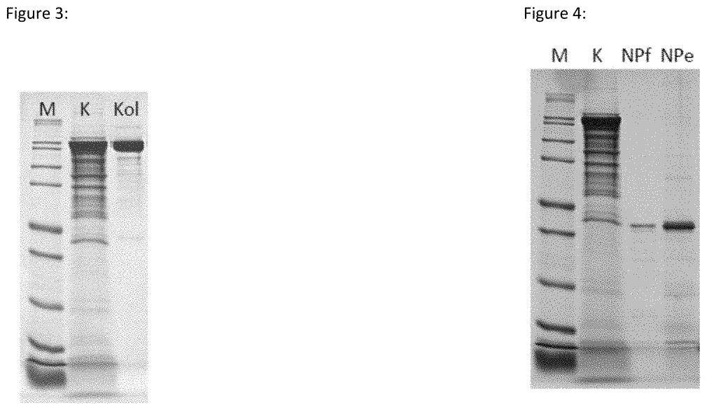

FIG. 3: SDS-PAGE of the collagenase value fraction after separation on polypropylene glycol (PPG-600M) (equivalent to fraction F6 in FIG. 1).
M: marker proteins; K: sample application concentrate (before purification/separation); Kol: Collagenase value fraction (F6).

FIG. 4: SDS-PAGE of the neutral protease value fraction after separation on polypropylene glycol (PPG-600M) (equivalent to fraction F7 in FIG. 1).
M: marker proteins; K: sample application concentrate (before purification/separation); NPf: Neutral protease value fraction (F7) after chromatography; NPe: Neutral protease after concentration of F7 to lyophilized final product.

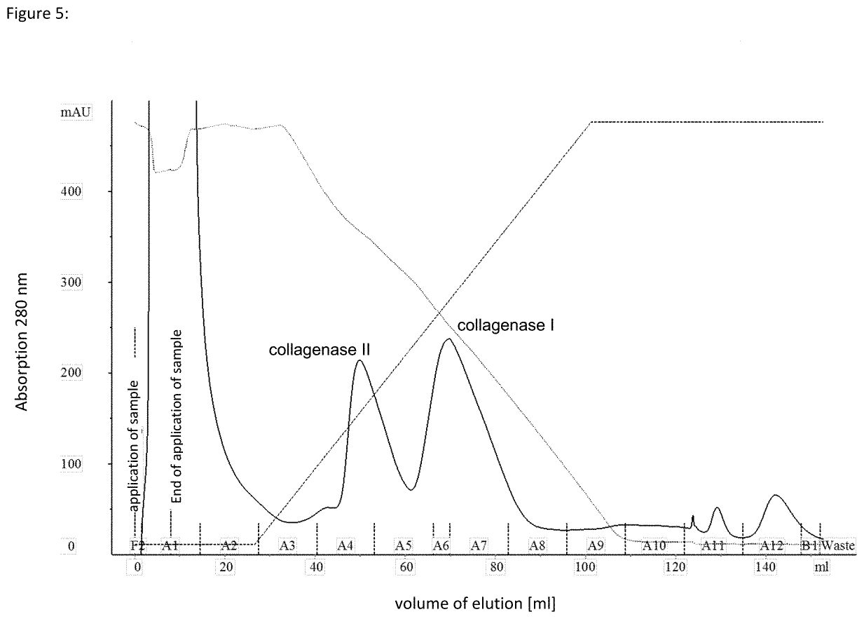

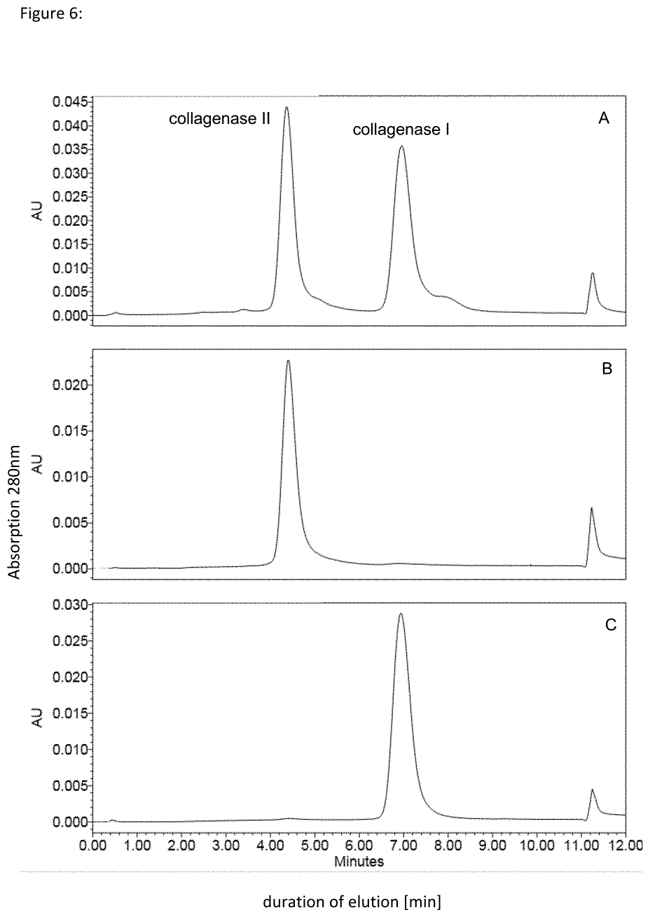

FIG. 5: Chromatogram of the purification and separation of the concentrate of the culture supernatant on butyl sepharose (Butyl Sepharose HP) showing the—compared to FIG. 1—additional separation of collagenase I and collagenase II.

FIG. 6: MonoQ chromatogram for comparative analysis of the respective collagenase value fractions after purification and separation of the cell-free, concentrated culture supernatant by HIC chromatography.
A: Collagenase value fraction after separation on polypropylene glycol (PPG-600M) (procedure variant 1); B: Collagenase type II value fraction after separation on butyl sepharose (Butyl Sepharose HP) (procedure variant 2); C: Collagenase type I value fraction after separation on butyl sepharose (Butyl Sepharose HP) (procedure variant 2).

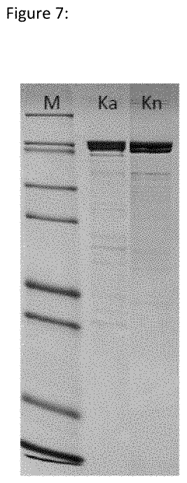

FIG. 7: SDS-PAGE for comparison of a currently commercially available collagenase (Ka) purified via several chromatographic steps with the collagenase (Kn) obtained in one chromatographic step according to method variant 2 of the invention.
M: Marker proteins

### EXAMPLES OF EMBODIMENTS

All mobile phases, application buffer, wash buffer and elution buffer used in the examples, are aqueous solutions. The complete ingredients of the mobile phases used are given below in each case.

### Example 1

A culture of Clostridium histolyticum was cultured to the desired cell density in liquid culture using a suitable nutrient medium according to standard methods. After separation of the cells by standard methods, such as centrifugation and/or filtration, the hydrophobic interaction chromatography of the method according to the invention was performed. For this purpose, a chromatography column (bed height about 20 cm) filled with polypropylene glycol (PPG-600M, Tosoh Bioscience LLC) was equilibrated by means of a mobile phase (aqueous solution, 0.85 mol/l ammonium sulfate, 20 mmol/l tris, 7 mmol/l CaCl₂, pH 7.5). After loading the cell-free concentrated culture supernatant, the column was washed with 10 column volumes (CV) of the same mobile phase. The elution of the target proteins was carried out at a linear flow rate of 250 cm/h in three elution steps. The first value fraction was obtained by isocratic elution with the aforementioned mobile phase and contained the clostripain. The second fraction was obtained by isocratic elution with another mobile phase (aqueous solution, 0.2 mol/l ammonium sulfate, 20 mmol/l tris, 7 mmol/l CaCl₂, pH 7.5) and contained the collagenases (collagenase I and collagenase II). The third fraction was obtained by isocratic elution with a third mobile phase (aqueous solution, 12% (m/m) propylene glycol, 20 mmol tris, 7 mmol CaCl₂, pH 7.5) and contained the neutral protease.

FIG. 1 shows a chromatogram of the above-mentioned purification and separation of the cell-free concentrated culture supernatant by means of hydrophobic interaction chromatography on PPG-600M as column material. The figure shows the value fractions of the three different products with the corresponding elution ranges. Clostripain elutes in subfractions F2 to F5, the collagenases in fraction F6, and neutral protease in fraction F7. The collagenase fraction (F6) contains both collagenases (type I and type II) in approximately equal proportions.

FIG. 2 shows the analysis of the collagenase value fraction after separation on PPG-600M (equivalent to fraction F6 in FIG. 1) using MonoQ chromatograms. Shown is the quality obtained with different column loadings in HIC chromatography (loading measured in volume-specific PZ activity according to Wunsch E., Heidrich H.-G.; Z. Physiol. Chem. 333: 149-151, 1963). For this purpose, a sample of the respective collagenase value fraction is analyzed using a MonoQ column (column: MonoQ 5/20 GL, GE Healthcare; application buffer: aqueous solution, 10 mmol/l tris, 2 mmol/l CaCl₂, pH 7.5; elution buffer: aqueous solution, 10 mmol/l tris, 2 mmol/l CaCl₂, 1 mol/l NaCl, pH 7.5; gradient elution). Thus, both the purity and the content of the collagenase types as well as their ratio to each other can be determined in each case. As can be seen from the chromatograms, the collagenase fraction obtained contains only minor impurities in addition to the two collagenase types, irrespective of the loading of the PPG column. The method is thus reproducible and robust. A value of >90% was determined for the purity of the collagenase fraction (see FIGS. 2 and 3).

Since no meaningful activity assay is currently available for collagenase I, its quality and quantity were evaluated using MonoQ analytics. As shown in FIG. 2, no significant degradation products can be detected in the MonoQ analytics of the collagenase I fraction, and the enzyme thus corresponds to the quality of the products available on the market.

FIG. 3 shows an SDS-PAGE of the collagenase value fraction after separation on PPG-600M (equivalent to fraction F6 in FIG. 1). This was carried out according to the protocol of U. K. Laemmli using a 14% tris glycine gel (Anamed) and stained with Coomassie (Coomassie R-250, Invitrogen) (Laemmli U. K.: Cleavage of structural proteins during the assembly of the head of bacteriophage T4; Nature 227: 680-685, 1970). In the left lane, labeled M, marker proteins (Novex Mark12, Invitrogen) are plotted. The middle lane, labeled K, is the cell-free, concentrated culture supernatant before purification and separation. In the right lane (Kol), the collagenase value fraction F6 from FIG. 1, containing collagenase I and collagenase II, was plotted. The figure thus shows a direct comparison of the purity of the collagenases before and after purification and separation on the PPG column. Although the collagenase proteins were purified and separated using only one chromatography column, the purity of the collagenase value fraction is >90%.

FIG. 4 shows a corresponding SDS-PAGE of the neutral protease value fraction after separation on PPG-600M (equivalent to fraction F7 in FIG. 1). Marker proteins were again applied to the left lane, labeled M. The middle lane, labeled K, is again the cell-free, concentrated culture supernatant before purification and separation. In the one of the two right lanes labeled NPf, the neutral protease value fraction F7 from FIG. 1 was applied directly after purification and separation on the PPG column. One of the two right lanes, labeled NPe, shows the neutral protease end product dissolved again for comparative analytics, which had previously been obtained from F7 by desalting, concentration and lyophilization (freeze-drying). It can be seen from FIG. 4 that the neutral protease also exhibits a high purity of well >80% after purification and separation on the PPG column. This is significantly higher than the purity of the neutral protease products currently available on the market.

Table 1 shows the respective relative yield of enzymatic activity after purification and separation of the enzymes from the cell-free, concentrated culture supernatant by means of hydrophobic interaction chromatography on PPG-600M as column material. Values of >90% were determined several times for the corresponding yield of collagenase II.

TABLE 1
Relative yield of enzymatic activity after purification and
separation of enzymes (results from several experiments).

| | Collagenase | Clostripain | Neutral |
|---|---|---|---|
| | type II (PZ)^{a} | (BAEE)^{b} | protease |
| Sample | (%) | (%) | (%) |
| Application | 100 | 100 | 100 |
| Flow (waste) | — | — | — |
| Value fraction clostripain | — | 40-80^{d} | — |
| Value fraction collagenase | 65-95 | 0-40^{d} | — |
| Value fraction | — | — | 60-100 |
| neutral protease | | | |

^{a}determined according to Wünsch E., Heidrich H.-G.; Z. Physiol. Chem. 333, 149-151, 1963
^{b}determined according to Mitchell W. M., Harrington W. F.; Methods Enzymol. 19: 635-642, 1970
c: determined according to Moore S., Stein W. H.; J. Biol. Chem. 176: 367-388, 1948
^{d}A portion is lost in the washing step with the waste fraction.

A special feature of the developed process is that the distribution of clostripain between a separate, pure clostripain fraction (comprising subfractions F2 to F5 in FIG. 1) and the collagenase fraction (fraction F6 in FIG. 1) can be controlled by how much column volume (CV) of the first elution buffer (clostripain elution buffer) is used to elute the chromatography column. A respective volume starting at about 12 CV leads to up to about 80% yield of total clostripain in the separate, pure clostripain fraction with corresponding losses with the waste fraction in the wash step and only very low clostripain content in the collagenase fraction. In contrast, a respective volume of approx. 4 CV, however, leads to a distribution of the total clostripain of approx. 40% in the separate, pure clostripain fraction and approx. 40% in the subsequent collagenase fraction, again with corresponding losses with the waste fraction in the washing step (see tables 1 and 2).

TABLE 2
Dependence of the amount of clostripain in the collagenase
value fraction on the volume of the first elution
buffer (clostripain elution buffer).

| Volume clostripain | Clostripain activity in the |
|---|---|
| elution buffer | collagenase value fraction |
| (CV) | (%) |
| 4 | approx. 40 |
| 7 | approx. 20 |
| 10 | <5 |
| 12 | <1 |

### Example 2

A culture of clostridium histolyticum was cultured to the desired cell density in liquid culture using a suitable nutrient medium according to standard methods. After separation of the cells by standard methods, such as centrifugation and/or filtration, the hydrophobic interaction chromatography of the method according to the invention was carried out. For this purpose, a chromatography column (bed height 20 cm) filled with butyl sepharose (Butyl Sepharose High Performance, abbreviated as Butyl Sepharose HP, GE Healthcare) was equilibrated by means of a mobile phase (aqueous solution, 2 mol/l KCl, 20 mmol/l tris, 7 mmol/l CaCl₂, pH 9). After application of the cell-free concentrated culture supernatant, the column was washed with 4 column volumes (CV) of the same mobile phase and eluted isocratically. The elution of the target proteins was carried out at a linear flow rate of 250 cm/h. The subsequent second elution step was carried out as a gradient over 20 CV with linearly decreasing salt concentration starting from the aforementioned mobile phase to the target buffer (aqueous solution, no KCl (0 mol/l), 20 mmol/l tris, 7 mmol/l CaCl₂, pH 9). The first value fraction hereby obtained contained the clostripain. The second fraction contained the collagenase II. The third fraction contained the collagenase I. The fourth fraction was obtained by a third elution step by isocratic elution with 5 CV of a further mobile phase (aqueous solution, 25% (m/m) propylene glycol, 20 mmol tris, 7 mmol CaCl₂, pH 9) and contained the neutral protease.

FIG. 5 shows a chromatogram of the above-described purification and separation of the cell-free, concentrated culture supernatant by hydrophobic interaction chromatography on butyl sepharose HP as column material. Shown is—in comparison to FIG. 1—the additional separation into collagenase I and collagenase II, wherein the first main peak in this respect corresponds to collagenase II and the second main peak in this respect corresponds to collagenase I.

FIG. 6 shows the comparative MonoQ analysis of the respective collagenase value fractions after purification and separation of the cell-free, concentrated culture supernatant by means of different variants of the hydrophobic interaction chromatography. A shows the collagenase value fraction after separation on PPG-600M as column material (method variant 1). B represents the collagenase type II value fraction after separation on butyl sepharose HP as column material (method variant 2). C shows the collagenase type I value fraction after separation on butyl sepharose HP as column material (method variant 2). Thus, a quality of purity and, if necessary, separation is achieved in one method step, which requires three or more method steps in the known methods.

FIG. 7 shows an SDS-PAGE after silver staining (Argent Quick Silver Staining Kit, Anamed). On the left lane, labeled M, the marker proteins were again applied. Next to it, for direct comparison, a currently commercially available “classical” collagenase purified over several chromatographic steps (middle lane, labeled Ka) and a collagenase obtained by a variant of the one-step purification method according to the invention (right lane, labeled Kn) are shown. It can be seen from the figure that the purity of the collagenases obtained by the one-step method according to the invention is at least as high as that of the “classical” collagenase currently available on the market. Due to the one-step method, the yield of ≥80% is also significantly higher than the yield of the established methods.

1-20. (canceled)

21. A method for the purification of at least one enzyme, selected from the group consisting of collagenase type I, collagenase type II, neutral protease and clostripain, from a mixture of substances, the method comprising:
subjecting the at least one enzyme to at least one hydrophobic interaction chromatography having a stationary phase that comprises a material selected from the group consisting of polypropylene glycol and butyl sepharose, and at least one aqueous solution as a mobile phase, the at least one aqueous solution comprising at least one salt selected from the group consisting of ammonium sulfate and potassium chloride.

22. The method according to claim 21, wherein the stationary phase comprises butyl sepharose and wherein at least one aqueous solution comprising ammonium sulfate is used as a mobile phase.

23. The method according to claim 22, wherein the stationary phase comprises butyl sepharose and wherein the at least one enzyme is eluted from the stationary phase with at least one aqueous solution comprising ammonium sulfate.

24. The method according to claim 21, wherein the method does not comprise a step of protein precipitation with ammonium sulfate.

25. The method according to claim 21, wherein the mixture of substances comprises at least two enzymes selected from the group consisting of collagenase type I, collagenase type II, neutral protease and clostripain.

26. The method according to claim 21, wherein the mixture of substances is a culture supernatant of Clostridium histolyticum.

27. The method according to claim 21, wherein the at least one aqueous solution has a molar concentration of ammonium sulfate of up to 1.5 mol/l.

28. The method according to claim 26, wherein the at least one aqueous solution has a molar concentration of ammonium sulfate in the range from 0.3 to 1.5 mol/l.

29. The method according to claim 21, wherein a step elution, a gradient elution or a combination of these two is carried out in the hydrophobic interaction chromatography.

30. The method according to claim 21, wherein a step elution is carried out in the hydrophobic interaction chromatography and at least three elution steps are carried out.

31. The method according to claim 30, wherein the two collagenases, neutral protease and clostripain are separated from one another, so that collagenase type I and collagenase type II are mixed in a common fraction and neutral protease and clostripain are separated from one another in two further fractions separate from the fraction comprising the two collagenases.

32. The method according to claim 30, wherein collagenase type I, collagenase type II, neutral protease and clostripain are separated from one another.

33. The method according to claim 21, wherein a gradient elution or a combination of gradient and step elution is carried out in the hydrophobic interaction chromatography and at least three elution steps are carried out.

34. The method according to claim 33, wherein the two collagenases, neutral protease and clostripain are separated from each other, so that collagenase type I and collagenase type II are mixed in a common fraction and neutral protease and clostripain are separated from each other and in two further fractions separate from the fraction comprising the two collagenases.

35. The method according to claim 33, characterized in that collagenase type I, collagenase type II, neutral protease and clostripain are separated from one another.

36. An enzyme obtained according to the method of claim 21.

37. A composition comprising at least one enzyme of claim 36.
